(19) United States
(12) Patent Application Publication
(10) Pub. No.: US 2009/0076439 A1
(43) Pub. Date: Mar. 19, 2009

(54) Inflation System for Balloon Catheter

(76) Inventors: Michael L. Dollar, Garland, TX (US); Vietor A. Dubuclet, IV, Dallas, TX (US); Thomas Dietzman, Arlington, TX (US)

Correspondence Address: CARSTENS & CAHOON, LLP, P O BOX 802334, DALLAS, TX, 75380, US

(21) Appl. No.: 11/855,840

(22) Filed: Sep. 14, 2007

(51) Int. Cl. A61M 25/10; A61M 29/04

(52) U.S. Cl. 604/28; 604/97.03

(57) ABSTRACT

The present invention is directed towards an inflation system for a balloon catheter that automatically determines and outputs a balloon diameter. The present invention is also directed towards an inflation system that automatically controls the surgical procedure using the balloon diameter.

### FIELD OF THE INVENTION

This invention relates to an inflation system for use with balloon catheters, and more specifically to an automatic inflation system for use with balloon catheters in the paranasal cavities of the sinus system.

### BACKGROUND OF THE INVENTION

In order to fully understand this invention, it is important to consider the anatomy of the sinus system. The sinus system consists of many different pathways, called ducts or ostia, which allow mucus, air and other substances to drain and flow through the system. Inflammation can occur in the tissues that make up the ducts and ostia, causing them to swell and block the normal flow. Inflammation may be caused by allergies, noxious agents, nasal polyps, and other factors. Over time there can be a pathologic increase in inflamed tissue causing permanent disruption in the flow through the sinus system. Obstruction of the narrow ducts and ostia between the paranasal sinuses and nasal cavity develops, resulting in a vicious cycle of increased secretions, edema and ultimately complete blockage of the sinus pathways. The state of chronic sinus inflammation is called sinusitis.

Treatment with antibiotics, corticosteroids in nasal sprays or systematically, and antihistamines may result in effective resolution of sinusitis. However, some patients become resistant to medical treatment and surgery becomes necessary. Endoscopic sinus surgery is performed from an intranasal approach, thus eliminating the need for external incisions. A type of minimally invasive surgery called balloon catheterization or sinuplasty can be used to effectively treat sinusitis while minimizing the amount of trauma experienced by the patient during and after surgery. Sinuplasty involves placing an expandable device, such as a deflated balloon, inside the clogged sinus pathways and inflating the balloon in order to open the clogged pathway. A fluoroscope, endoscope or image guided surgery system is typically used to place the balloon in the proper position.

Once the balloon catheter is in place inside the clogged pathway, the balloon is inflated in order to open the clogged pathway. Typically balloon inflation is accomplished by injecting a fluid into the balloon catheter from a syringe. In the prior art, the syringe is controlled manually by the physician or technician. Care must be taken to ensure that the pressure inside the balloon catheter does not exceed the burst pressure of the balloon, which is difficult to accomplish manually. Additionally, in the prior art the diameter of the inflated balloon, and thereby the diameter of the affected sinus pathway, is determined by visual observation through a fluoroscope or other image guided surgery system. Visual inspection of the balloon is difficult in the nasal cavity once the balloon has inflated, and even when visual inspection is possible it is difficult to accurately determine the diameter by sight alone due to the lens curvature of the endoscope creating a non-linear cross-sectional view. Thus, there exists a need in the art to easily and automatically monitor and control the balloon catheter pressure. There further exists a need in the art for a way to accurately determine the diameter of the balloon inside the nasal system, and thus the size of the sinus pathway being operated on, without relying on visual inspection.

The pathways of the nasal system present special problems in this area. Whereas the blood vessels in the cardiovascular system are uniformly relatively compliant during balloon expansion, the tissues that make up the nasal system are more complex. The nasal system is comprised of a relatively compliant tissue called mucosa, and relatively non-compliant cartilage and bone tissues. The complex nature of nasal tissue makes the task of determining balloon diameter a difficult one. The present invention is thus directed towards a method and system that overcomes these difficulties.

### SUMMARY OF THE INVENTION

The present invention is thus directed towards an inflation system for a balloon catheter that automatically monitors and controls the pressure inside the balloon catheter, and which accurately determines the diameter of the inflated balloon inside the nasal cavity without relying on visual inspection of the balloon.

In one embodiment of the present invention, an electronically monitored and controlled inflation system is provided. The inflation system continuously monitors the fluid pressure inside the balloon catheter and the volume of fluid infused into the balloon catheter. A signal processor integral to the inflation system compares this measured data with an empirically determined relationship between pressure, volume and balloon diameter to calculate a balloon diameter during treatment. In one embodiment, the signal processor determines and outputs to a display means the size of the nasal passageway prior to treatment by inflating the balloon to between about 1 and about 5 atmospheres, and detecting the inflection point at which the balloon fully contacted the walls of the passageway. In another embodiment, the signal processor determines and outputs to a display means the diameter of the balloon during treatment. In yet another embodiment, the signal processor automatically controls the procedure by automatically inflating the balloon to a predetermined diameter for a predetermined period of time.

### BRIEF DESCRIPTION OF THE DRAWINGS

A more complete understanding of the method of the present invention may be had by reference to the following detailed description when taken in conjunction with the accompanying drawings, wherein:

FIG. 1 is a perspective view of one embodiment of the present invention;

FIG. 2 is an example graph of isodiametric pressure versus infused volume plots and fitted curves;

FIG. 3 is an example graph of isobaric balloon diameter versus infused volume curves;

FIG. 4 is an example graph of a pressure versus volume curve for a balloon catheter inflated in an obstructed nasal passageway;

FIG. 5 is a flowchart for the method of the present invention;

Where used in the various figures of the drawing, the same numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

The present invention is directed towards an inflation system for balloon catheters used to treat obstructed pathways in the nasal or sinus system. The nasal system is comprised of a mixture of relatively compliant tissue called mucosa and relatively non-compliant bone and cartilage tissues. During balloon catheter surgery, the mucosa layer of tissue is typically the first layer contacted by the balloon as it inflates. As the balloon inflates, this relatively compliant tissue layer compresses under the pressure of the balloon. Once the mucosa layer has been fully compressed, the less compliant bone and/or cartilage tissues start to move and compress. However, these less compliant tissues that underlie the mucosa require more pressure imparted onto them by the balloon in order to move and compress. The inflation system of the present invention is able to determine the diameter of a balloon catheter as it is being inflated inside an obstructed nasal pathway made of a mixture of relatively compliant and relatively non-compliant tissue.

The inflation system of the present invention automatically monitors and controls the fluid pressure and fluid volume inside the balloon catheter, which in turn controls the balloon diameter. The inflation system is also able to execute a programmed set of inflation/deflation times and cycles. The invention will be described with reference to a particular embodiment, but variations on the particular embodiments described herein below do not depart from the spirit and scope of the present invention in its broadest sense.

Generally, the inflation system of the present invention comprises a balloon inflator, a balloon catheter, a fluid pressure transducer, a signal processor and a display means. The signal processor receives signals from the fluid pressure transducer and the balloon inflator, determines the fluid pressure and volume of fluid infused into the balloon catheter, calculates an estimated balloon diameter based on a known relationship between fluid pressure, volume and balloon diameter for the particular balloon catheter being used, and outputs to the display means the balloon diameter.

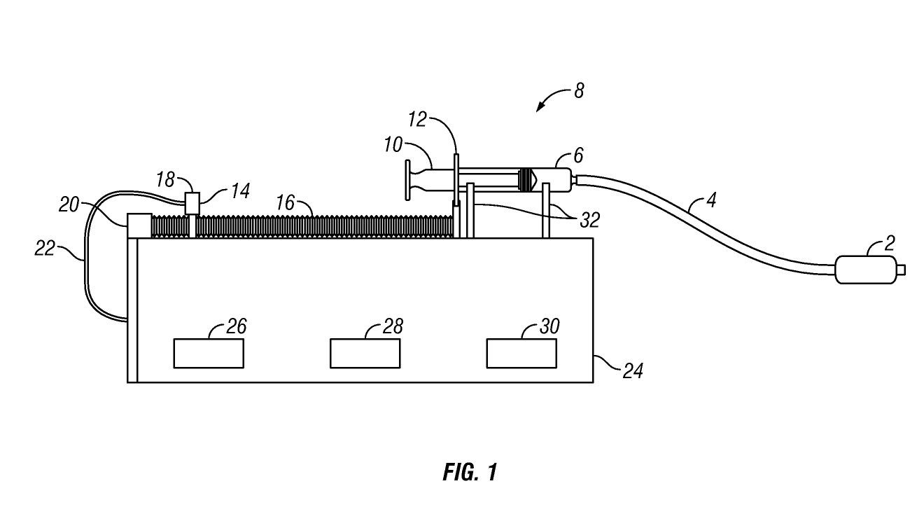

Referring initially to FIG. 1, therein is depicted one embodiment of the present invention. A base 24 is provided to house and support the electrical and mechanical components of the present invention. The base 24 can be made of metal, plastics or other sufficiently rigid material. Electrical components such as circuit boards, transformers, wires and other components designed to receive, transmit and process electromagnetic signals are housed within the base.

In one embodiment, the base 24 incorporates one or more display means 26, 28, 30 for selectively outputting visual display of predetermined parameters. For example, the pressure inside the balloon catheter, the time period the balloon catheter has been inflated, and the number of inflation cycles can be displayed on one or more display means 26, 28, 30. The display means is preferably incorporated into or mounted on a side of the base 24 and wired to receive signals from the electrical components within the base of the inflation system. However, the display means can be detached from the base 24 as long as it is in electronic communication with the rest of the inflation system. The display means can also be a liquid crystal display, light emitting diode display, cathode ray tube, plasma display or other feasible display screen known in the art.

On the top of the base 24 is the syringe mount 32. A syringe 8 that can be used with the present invention comprises a syringe barrel 6, which is typically molded from a relatively transparent plastic material to permit visual inspection of the syringe contents. Although a syringe 8 is the particular type of balloon inflator used in one embodiment of the present invention, other types of pumps can be used as a balloon inflator without departing from the spirit and scope of the present invention. For example, a pressurized fluid bag can also be used as a balloon inflator.

In one embodiment, the syringe barrel 6 comprises at least one flange 8 protruding from the proximal end of the barrel 6. A syringe plunger 10 fits inside the syringe barrel and can slide back and forth between the proximal end and distal end of the barrel 6. A soft rubber bulb is typically provided at the proximal end of the syringe plunger 10, which engages the interior of the barrel 6 in a fluid tight fit such that sliding the syringe plunger 10 towards the distal end of the syringe barrel 6 exerts positive pressure on the fluid inside the syringe barrel 6. Similarly, sliding the syringe plunger 10 towards the proximal end of the barrel 6 reduces the fluid pressure inside the syringe barrel 6.

The syringe mount 32 is adapted to support and retain the syringe barrel 6. In one embodiment, the syringe mount 32 is adapted to receive at least one of the flanges 12 protruding from the proximal end of the syringe barrel 6. Regardless of actual construction, the syringe mount 32 is designed to hold the syringe barrel 6 in one position during the surgery and then allow the syringe 8 to be removed from the syringe mount 32 and discarded after surgery is complete. Various constructions of the syringe mount 32 are within the skill of the art. While the syringe barrel 6 is mounted to the syringe mount 32, the syringe plunger 10 should be able to move freely between the proximal end and the distal end of the barrel 6.

Also on top of the inflation system base is a plunger actuator. In one embodiment, the plunger actuator comprises a helically threaded cylindrical member 16, a motor 20 and a hammer 18. The hammer 18 attaches to, presses against or engages the proximal end of the plunger 10. The hammer 18 comprises a threaded hole that is designed to engage the threads of the threaded member 16, whereby rotating the threaded member 16 about its axis moves the hammer 18 linearly in the threaded member's axial direction. To accomplish linear motion of the hammer 18, rotation of the hammer 18 about the threaded member's axis must be inhibited. A motor 20 is provided to rotate the threaded member 16 about its axis, which can be any electric motor known in the art. Preferably, the motor 20 is able to rotate the threaded member 16 both ways about its axis at variable speed in response to electrical control signals. Alternatively, the hammer 18 can be moved in a linear direction using a hydraulic actuator, which is within the skill of the art. Regardless of actual construction, a plunger actuator is provided that is capable of moving the plunger 10 between the distal and proximal ends of the syringe barrel 16 in response to electrical control signals.

In one embodiment, a force transducer 14 is also provided on the surface of the plunger actuator that engages the syringe plunger. The force transducer 14 senses the force applied to the plunger actuator by the plunger 10 and outputs an electromagnetic signal proportional to the sensed force. The force measured by the force transducer 14 is referred to herein as the “plunger force”. The transducer function can be accomplished by a variety of devices, for example, a piezoresistive semiconductor transducer, a fiber optic substrate emitting light at frequencies that are proportional to the force being applied to the substrate, or a radio transmitter in electrical communication with a pressure sensitive substrate for which changes in modulated frequencies are proportional to the pressures being applied to the substrate. The electrical signal output from the transducer is preferably transmitted to a converter, which converts it to a digital signal that can be processed by the signal processor inside the inflation system housing.

The fluid pressure inside the syringe can be determined by converting the plunger force measured by the force transducer to the fluid pressure. In one embodiment, the fluid pressure is determined by dividing the plunger force by the surface area of the soft rubber bulb at the proximal end of the plunger that is actually in contact with the fluid inside the syringe. Other methods of converting the plunger force to fluid pressure can be used, for example, by comparing the measured plunger force with conversion tables that are compiled prior to surgery.

The force conversion is carried out by a signal processor. In one embodiment, the signal processor is a microcomputer. The signal processor includes all of the necessary interface circuitry, circuit boards, silicon chips and wiring needed to receive, control and process input signals, and transmit display signals to at least one display means provided on the inflation system base and control signals sent to the plunger actuator. In one embodiment, the signal processor also includes a buffer, amplifier or filter to condition the plunger force signal received from the force transducer.

A cable 22 is provided to transmit the measured plunger force signal to the signal processor located inside the base 24. After it receives the force signal, the signal processor converts the force signal to a fluid pressure signal as described above. This method of converting the plunger force into fluid pressure is an indirect method of measuring fluid pressure. Alternatively, the fluid pressure can be measured directly by placing a fluid pressure transducer (not shown) in fluid communication with the syringe and balloon catheter. This direct measurement method has advantages and disadvantages in comparison to the indirect method outlined above. While directly measuring the fluid pressure may yield a slightly more accurate fluid pressure reading, the practitioner also has to worry about the syringe fluid coming into contact with more surfaces and devices, which raises more sterility issues. By the same token, the indirect method may be slightly less accurate than the direct method, but the simplicity involved in changing out the syringe and balloon catheter without having to connect a fluid pressure transducer in fluid communication with them is a distinct advantage.

Regardless of how the fluid pressure is measured, the signal processor also uses the fluid pressure to determine the balloon diameter or circumference, which in turn tells the surgeon the size of the passageway being operated on. In one embodiment, the method used by the signal processor to make the conversion to balloon diameter is to compare the fluid pressure with a reference array stored in the computer. In one embodiment, the reference array is pre-programmed into the signal processor. In another embodiment, the reference array is input into the signal processor by the practitioner prior to surgery. In another embodiment, the signal processor uses an analytical algorithm to determine the balloon diameter.

Any method used to calculate diameter will involve a predetermined relationship between the fluid pressure, the volume of fluid injected into the balloon catheter, and balloon diameter. Some methods in the prior art rely on fluid pressure/balloon diameter relationships that are determined by inflating the balloon under ideal, or compliant, conditions, giving a pressure versus diameter curve that has a smooth, relatively constant slope. Generally, these smooth curves are helpful and relevant to balloon catheters used to perform balloon catheterization in obstructed veins and arteries in the cardiovascular system. Veins and alteries are fairly compliant and easily stretched throughout the balloon inflation process, which makes the relationship between fluid pressure and volume a fairly smooth curve when it is graphed. Obstructed sinus pathways, however, behave differently.

The sinus system is comprised primarily of relatively non-compliant cartilage and bone tissues that underlie a relatively compliant layer of mucosa tissue. Moreover, when a practitioner is using balloon catheterization to open up obstructed nasal passageways, the relatively non-compliant tissues must be moved and compressed for the surgery to be successful. It has been experimentally determined that all of the relatively non-compliant bone and cartilage tissues present in the sinus system will be fully moved and compressed when the pressure on them has reached at least 8 atmospheres.

It has also been experimentally determined that, for a balloon that is inflated in an obstructed sinus pathway, the slope of a pressure versus volume infused into the balloon catheter is not a smooth curve, but instead typically involves four phases of balloon expansion. FIG. 4 is an example graph showing the four distinct phases that occur during balloon inflation inside an obstructed sinus pathway. During a first phase I of balloon inflation, the walls of the balloon expand inside the obstructed sinus pathway until the walls of the balloon are flush against the walls of the obstructed pathway. Typically, the walls in the sinus system are comprised of the relatively compliant mucosa tissue. The balloon inflation then commences a second phase II of balloon expansion, whereby the pressure inside the balloon rises more quickly in relation to volume infused. This second phase II of balloon expansion is due to the fact that the relatively compliant mucosa sinus tissue is resisting the expansion of the balloon somewhat, and requires higher pressure to compress. At the conclusion of the second phase and the beginning of the third phase III, the relatively compliant mucosa tissue has been fully compressed, and the relatively non-compliant bone and cartilage tissue in the obstructed nasal passageway begins to compress. During this third phase III, the bone and cartilage tissue move and compress so that the obstructed nasal passageway can be effectively opened. The compression of this relatively non-compliant tissue resists balloon expansion even more than the relatively compliant mucosa tissue, which means the pressure rises even more sharply in relation to infused volume. The third phase III concludes and the fourth phase IV begins when the relatively non-compliant tissues have moved and compressed to the fullest extent possible and the nasal passageway has been fully expanded. Increasing the pressure inside the balloon beyond this point does little to increase balloon diameter. During the fourth phase IV, the slope of the pressure versus volume relationship is at its steepest.

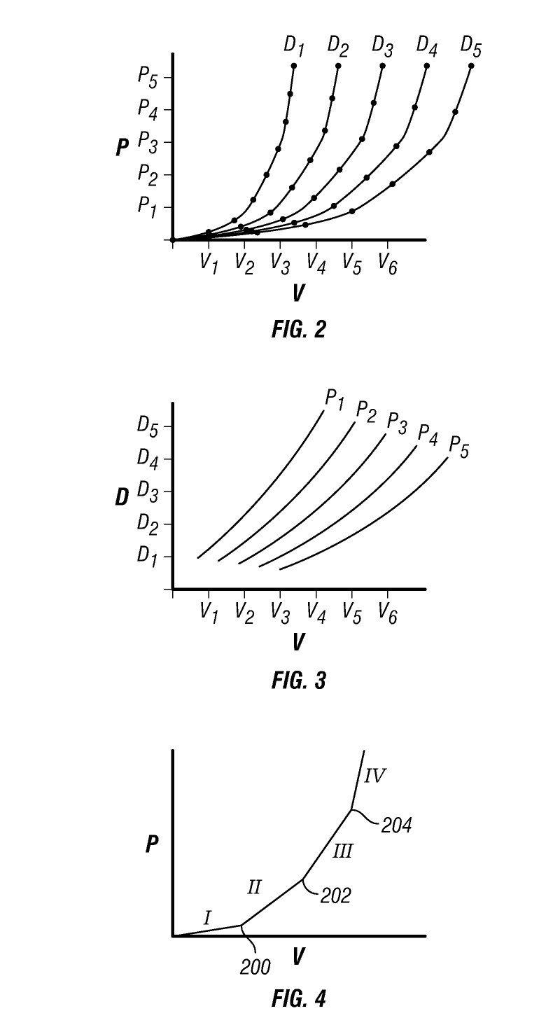

The balloon diameter can be determined using a predetermined or known relationship between the fluid pressure, infused volume, and balloon diameter. This relationship can be determined by plotting pressure versus infused volume while a balloon catheter is inserted into holes of a known diameter that have been drilled into a completely non-compliant material. An example plot of such a relationship is shown in FIG. 2. The pressure/volume/diameter relationship for a balloon having a known maximum diameter can be determined by drilling a predetermined number of holes of known diameters into an aluminum block. Referring to FIG. 2, the holes could range in diameter from D1 to D5. The balloon could be first inserted into the D1 hole and inflated from about 0 atmospheres to about P5 atmospheres. During inflation, the pressure and infused volume can be continuously or periodically measured and plotted. The process can then be repeated for each successive hole, D2 through D5, producing a number of isodiametric pressure versus infused volume plots. A curve fitting technique, such as polynomial curve fitting, can then be used to formulate isodiametric equations that relate pressure to infused volume.

During surgery, as the balloon is being inflated, the fluid pressure and infused volume are continuously or periodically measured by the inflation system. The fluid pressure inside the balloon can be measured as described herein above, but can be measured by any pressure transducer known in the art. The volume can be calculated, in the case of a syringe, using the known diameter of the syringe barrel and the length through which the plunger has traveled. In another embodiment, a volumetric flow meter is provided in the fluid stream to measure infused volume. In its broadest sense, any method known in the art to measure the volume of fluid infused into the balloon catheter can be used.

The isodiamatric equations formulated above can then be used to determine the relationship between balloon diameter and infused volume. Again, when balloon catheter surgery is being performed in accordance with the present invention, the fluid pressure and volume of infused fluid are measured. For the particular measured fluid pressure, an isobaric relationship between balloon diameter and volume of infused fluid can be determined from the isodiametric equations referred to above. FIG. 3 is an example graph of several isobaric curves representing the relationship between balloon diameter and infused volume for pressures P1 through P5. For example, if the fluid pressure inside the balloon is measured at P1, an isobaric relationship between balloon diameter and infused volume can be constructed for a pressure of P1. The isobaric relationship is constructed by calculating the infused volume using each isodiametric equation for D1 through D5 at P11 and plotting that infused volume against each balloon diameter D1 through D5. The result will be an isobaric diameter versus infused volume data plot for P1, which can then be used to formulate, for example, a polynomial curve that will allow the signal processor to calculate an estimated balloon diameter for the particular pressure P1 and measured infused volume. Other curve fitting techniques could be used.

Once the balloon diameter is determined as described above, the signal processor outputs a signal to a display means so the surgeon can monitor the progress of the balloon catheter surgery without having to visually inspect the balloon. The calculated diameter can also be used by the signal processor to automate the surgery procedure. In one embodiment, the signal processor is programmed to inflate the balloon to a specific diameter, hold the balloon at that diameter for a predetermined period of time, deflate the balloon back down so the balloon catheter can be removed. In another embodiment, the inflation/hold/deflation cycle is repeated automatically by the signal processor. As stated previously, it has been experimentally determined that the balloon pressure must reach about 8 atmospheres to compress the non-compliant sinus tissues and fully treat an obstructed sinus pathway. In another embodiment, the signal processor is also programmed to stop the balloon inflation when the fluid pressure inside the balloon catheter is nearing the burst pressure of the balloon inflation device.

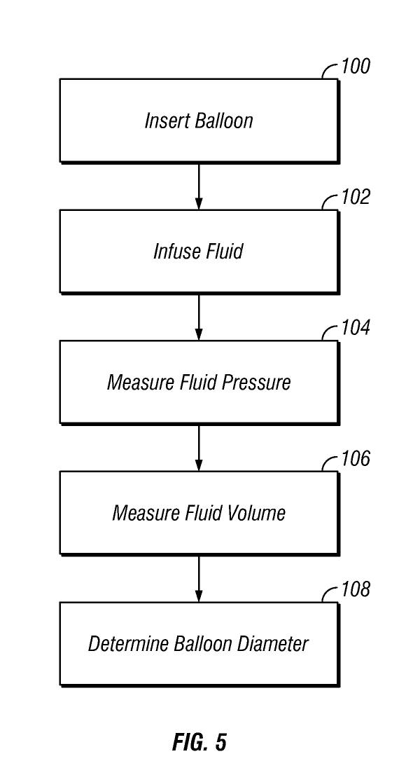

The general method steps of the present invention are represented in FIG. 5. First, the balloon is inserted 100 into an obstructed nasal passageway. Next, fluid is infused 102 into the balloon. During infusion, the fluid pressure and volume of infused fluid are measured 104, 106. Finally the diameter of the balloon is determined 108 as described above.

The inflation system of the present invention can also be used to determine the diameter of the obstructed nasal passageway prior to treatment. This function can be useful as a presurgical diagnostic tool. In one embodiment, the balloon is inflated to a pressure between about 1 atmosphere and about 5 atmospheres in order to determine the size of the nasal passageway prior to it being treated. Preferably, the balloon is only inflated to between about 1 and about 2 atmospheres. The diameter of the balloon is determined by identifying the first inflection point on the graph of balloon pressure versus infused volume. The inflection point occurs between the first phase of inflation, wherein the balloon is inflating inside the nasal passageway and encountering very little resistance to inflation, and the second phase, wherein the outer surface of the inflating balloon is in full contact with the inner surface of the nasal passageway being treated and the relatively compliant mucosa tissue in the nasal passageway is beginning to expand. The pressure and infused volume at that inflection point can then be used as described above to determine the balloon diameter, and thus the untreated nasal passageway diameter. The signal processor determines the inflection point by continuously monitoring the slope of the pressure versus volume plot. The inflection point occurs when the signal processor identifies a sharp increase in slope. Once the inflection point is identified, the inflation stops, the balloon is deflated and the balloon diameter at the inflection point is output to the display means.

The inflation system of the present invention can also be used to indicate to the practitioner when the balloon catheter has started to move and compress the relatively non-compliant bone and cartilage tissue that underlies the mucosa layer. For this method, the signal processor is adapted to detect the inflection point between the second phase and third phase of balloon expansion. Again, when the relatively compliant mucosa layer has been fully compressed and the balloon starts to move and compress the relatively non-compliant cartilage and bone tissue, the slope of the pressure versus volume curve sharply increases. By continuously or periodically measuring both pressure and volume, the signal processor can determine when this change in slope occurs and output a signal to the display means indicating that bone and cartilage tissue have begun to move and compress.

We claim:

1. A balloon catheter inflation system comprising:
a balloon inflator;
a display means;
a balloon catheter in fluid communication with said balloon inflator;
a fluid pressure transducer adapted to measure fluid pressure inside said balloon catheter;
a signal processor adapted to:
receive electrical signals from said balloon inflator which indicate volume of fluid infused into said balloon catheter;
receive electrical signals from said fluid pressure transducer which indicate fluid pressure inside said balloon catheter;
determine the volume of fluid infused into said balloon catheter and the fluid pressure inside said balloon catheter;
determine balloon diameter by comparing the volume of fluid infused and the fluid pressure with a predetermined relationship between fluid pressure volume infused and balloon diameter;
output electrical signals which indicate said balloon diameter to said display means.

2. The inflation system of claim 1 wherein said balloon inflator is a syringe comprising:
a syringe barrel having an interior, a distal end and a proximal end;
a plunger adapted to engage the interior of the syringe barrel in a fluid tight fit such that sliding the plunger towards the distal end of the syringe barrel exerts positive pressure on any fluid inside the barrel and sliding the plunger towards the proximal end of the syringe barrel exerts negative pressure on any fluid inside the barrel.

3. The inflation system of claim 1 wherein said display means is at least one of a liquid crystal display, a light emitting diode, a cathode ray tube, or a plasma display.

4. The inflation system of claim 2 wherein said fluid pressure transducer is a force transducer adapted to detect force imparted on said plunger.

5. The inflation system of claim 2 wherein said fluid pressure transducer is in fluid communication with said balloon inflator and said balloon catheter.

6. The inflation system of claim 2 further comprising a syringe mount adapted to hold said syringe barrel in place while allowing said plunger to move between said proximal end and said distal end of said syringe barrel.

7. The inflation system of claim 1 wherein said fluid pressure transducer is at least one of a piezoresistive semiconductor transducer, a fiber optic substrate emitting light at frequencies that are proportional to force being applied to the substrate, or a radio transmitter in electrical communication with a pressure sensitive substrate for which changes in modulated frequencies are proportional to pressures being applied to the substrate.

8. The inflation system of claim 1 wherein said predetermined relationship between fluid pressure, volume infused and balloon diameter comprises:
at least two isodiametric relationships between fluid pressure and volume infused; and
at least one isobaric relationship between balloon diameter and volume infused.

9. The inflation system of claim 1 further comprising an incompressible fluid inside said balloon catheter.

10. A balloon catheter surgical method for operating on a human body with at least one obstructed nasal pathway, comprising the steps of:
providing a balloon catheter comprising an inflatable balloon;
providing a balloon inflator in fluid communication with said balloon catheter;
inserting said balloon catheter into said obstructed nasal pathway;
infusing a fluid into said balloon catheter;
measuring fluid pressure inside said balloon catheter;
measuring volume of said fluid infused into said balloon catheter from said balloon inflator;
determining a diameter of said inflatable balloon by comparing said fluid pressure and said volume of fluid infused to a predetermined relationship between fluid pressure, volume of fluid infused and balloon diameter.

11. The method of claim 10 wherein said predetermined relationship between fluid pressure, volume of fluid infused and balloon diameter comprises:
at least two isodiametric relationships between fluid pressure and volume infused; and
at least one isobaric relationship between balloon diameter and volume infused.

12. The method of claim 11 wherein said determining a diameter of said inflatable balloon further comprises:
inputting said measured volume of fluid infused into the isobaric relationship corresponding to said measured fluid pressure; and
outputting a balloon diameter.

13. The method of claim 10 further comprising:
halting said infusing of fluid into said balloon catheter when said fluid pressure reaches a pressure between 1 atmosphere and 5 atmospheres;
determining an inflection point in the relationship between fluid pressure and volume of fluid infused;
using the fluid pressure and volume of fluid infused measured at said inflection point for said determining of said diameter of said inflatable balloon.

14. The method of claim 13 wherein said halting occurs when said fluid pressure reaches a pressure between 1 atmosphere and 2 atmospheres.

15. The method of claim 10 further comprising outputting said diameter of said inflatable balloon to at least one display means.

16. The method of claim 10 wherein said balloon inflator comprises:
a syringe barrel having an interior, a distal end and a proximal end;
a plunger adapted to engage the interior of the syringe barrel in a fluid tight fit such that sliding the plunger towards the distal end of the syringe barrel exerts positive pressure on any fluid inside the barrel and sliding the plunger towards the proximal end of the syringe barrel exerts negative pressure on any fluid inside the barrel.

17. The method of claim 10 further comprising controlling said infusing of fluid into said balloon catheter based on said inflatable balloon diameter.

18. The method of claim 10 wherein said measuring of said fluid pressure is performed continuously or periodically.

19. The method of claim 10 wherein said measuring of said volume of infused fluid is performed continuously or periodically.

20. The method of claim 10 wherein said determining of said diameter is performed continuously or periodically.

21. The method of claim 10 wherein said fluid is an incompressible fluid.
